SEQUENCE LISTING (44 sequences)

SEQ ID NO: 1 - DNA, 20 bp, Artificial Sequence
  forward primer for amplification specific fragments of IS6110 gene in Mycobacterium tuberculosis complex
cgaactcaaggagcacatca

SEQ ID NO: 2 - DNA, 20 bp, Artificial Sequence
  reverse primer for amplification specific fragments of IS6110 gene in Mycobacterium tuberculosis complex
agtttggtcatcagccgttc

SEQ ID NO: 3 - DNA, 17 bp, Artificial Sequence
  forward primer for amplification specific fragments of 16S rRNA gene in Nontuberculous mycobacteria
ggyrayctgccctgcac

SEQ ID NO: 4 - DNA, 18 bp, Artificial Sequence
  reverse primer(NTM-1) for amplification specific fragments of 16S rRNA gene in Nontuberculous mycobacteria
cccacaccgcaaaagctt

SEQ ID NO: 5 - DNA, 17 bp, Artificial Sequence
  reverse primer(NTM-1) for amplification specific fragments of 16S rRNA gene in Nontuberculous mycobacteria
cccacaccgcaaaagct

SEQ ID NO: 6 - DNA, 18 bp, Artificial Sequence
  reverse primer(NTM-1) for amplification specific fragments of 16S rRNA gene in Nontuberculous mycobacteria
tcccacaccgcaaaagct

SEQ ID NO: 7 - DNA, 18 bp, Artificial Sequence
  reverse primer(NTM-2) for amplification specific fragments of 16S rRNA gene in Nontuberculous mycobacteria
catcccacaccgctaccw

SEQ ID NO: 8 - DNA, 17 bp, Artificial Sequence
  forward primer for amplification specific fragments of 16S rRNA gene in Nontuberculous mycobacteria
ggtaatctgccctgcac

SEQ ID NO: 9 - DNA, 17 bp, Artificial Sequence
  forward primer for amplification specific fragments of 16S rRNA gene in Nontuberculous mycobacteria
ggtaacctgccctgcac

SEQ ID NO: 10 - DNA, 17 bp, Artificial Sequence
  forward primer for amplification specific fragments of 16S rRNA gene in Nontuberculous mycobacteria
ggcaatctgccctgcac

SEQ ID NO: 11 - DNA, 17 bp, Artificial Sequence
  forward primer for amplification specific fragments of 16S rRNA gene in Nontuberculous mycobacteria
ggcaacctgccctgcac

SEQ ID NO: 12 - DNA, 17 bp, Artificial Sequence
  forward primer for amplification specific fragments of 16S rRNA gene in Nontuberculous mycobacteria
ggtgatctgccctgcac

SEQ ID NO: 13 - DNA, 17 bp, Artificial Sequence
  forward primer for amplification specific fragments of 16S rRNA gene in Nontuberculous mycobacteria
ggtgacctgccctgcac

SEQ ID NO: 14 - DNA, 17 bp, Artificial Sequence
  forward primer for amplification specific fragments of 16S rRNA gene in Nontuberculous mycobacteria
ggcgatctgccctgcac

SEQ ID NO: 15 - DNA, 17 bp, Artificial Sequence
  forward primer for amplification specific fragments of 16S rRNA gene in Nontuberculous mycobacteria
ggcgacctgccctgcac

SEQ ID NO: 16 - DNA, 18 bp, Artificial Sequence
  reverse primer for(NTM-2) amplification specific fragments of 16S rRNA gene in Nontuberculous mycobacteria
catcccacaccgctacct

SEQ ID NO: 17 - DNA, 18 bp, Artificial Sequence
  reverse primer for(NTM-2) amplification specific fragments of 16S rRNA gene in Nontuberculous mycobacteria
catcccacaccgctacca

SEQ ID NO: 18 - DNA, 20 bp, Artificial Sequence
  forward primer for amplification specific fragments of IS6110 gene in Mycobacterium tuberculosis complex
agaaggcgtactcgacctga

SEQ ID NO: 19 - DNA, 20 bp, Artificial Sequence
  reverse primer for amplification specific fragments of IS6110 gene in Mycobacterium tuberculosis complex
ctgaaccggatcgatgtgta

SEQ ID NO: 20 - DNA, 22 bp, Artificial Sequence
  forward primer for amplification specific fragments of 16S rRNA gene in Nontuberculous mycobacteria
catgtyttstggkgsaaagctt

SEQ ID NO: 21 - DNA, 19 bp, Artificial Sequence
  reverse primer for amplification specific fragments of 16S rRNA gene in Nontuberculous mycobacteria
cgtaggagtctgggccgta

SEQ ID NO: 22 - DNA, 22 bp, Artificial Sequence
  forward primer for amplification specific fragments of 16S rRNA gene in Nontuberculous mycobacteria
catgtcttgtgggggaaagctt

SEQ ID NO: 23 - DNA, 22 bp, Artificial Sequence
  forward primer for amplification specific fragments of 16S rRNA gene in Nontuberculous mycobacteria
catgttttgtgggggaaagctt

SEQ ID NO: 24 - DNA, 22 bp, Artificial Sequence
  forward primer for amplification specific fragments of 16S rRNA gene in Nontuberculous mycobacteria
catgtcttctgggggaaagctt

SEQ ID NO: 25 - DNA, 22 bp, Artificial Sequence
  forward primer for amplification specific fragments of 16S rRNA gene in Nontuberculous mycobacteria
catgtcttgtggtggaaagctt

SEQ ID NO: 26 - DNA, 22 bp, Artificial Sequence
  forward primer for amplification specific fragments of 16S rRNA gene in Nontuberculous mycobacteria
catgtcttgtggggcaaagctt

SEQ ID NO: 27 - DNA, 22 bp, Artificial Sequence
  forward primer for amplification specific fragments of 16S rRNA gene in Nontuberculous mycobacteria
catgttttctgggggaaagctt

SEQ ID NO: 28 - DNA, 22 bp, Artificial Sequence
  forward primer for amplification specific fragments of 16S rRNA gene in Nontuberculous mycobacteria
catgtcttctggtggaaagctt

SEQ ID NO: 29 - DNA, 22 bp, Artificial Sequence
  forward primer for amplification specific fragments of 16S rRNA gene in Nontuberculous mycobacteria
catgtcttgtggtgcaaagctt

SEQ ID NO: 30 - DNA, 22 bp, Artificial Sequence
  forward primer for amplification specific fragments of 16S rRNA gene in Nontuberculous mycobacteria
catgttttgtggggcaaagctt

SEQ ID NO: 31 - DNA, 22 bp, Artificial Sequence
  forward primer for amplification specific fragments of 16S rRNA gene in Nontuberculous mycobacteria
catgtcttctggggcaaagctt

SEQ ID NO: 32 - DNA, 22 bp, Artificial Sequence
  forward primer for amplification specific fragments of 16S rRNA gene in Nontuberculous mycobacteria
catgttttgtggtggaaagctt

SEQ ID NO: 33 - DNA, 22 bp, Artificial Sequence
  forward primer for amplification specific fragments of 16S rRNA gene in Nontuberculous mycobacteria
catgttttctggtggaaagctt

SEQ ID NO: 34 - DNA, 22 bp, Artificial Sequence
  forward primer for amplification specific fragments of 16S rRNA gene in Nontuberculous mycobacteria
catgttttctggggcaaagctt

SEQ ID NO: 35 - DNA, 22 bp, Artificial Sequence
  forward primer for amplification specific fragments of 16S rRNA gene in Nontuberculous mycobacteria
catgttttgtggtgcaaagctt

SEQ ID NO: 36 - DNA, 22 bp, Artificial Sequence
  forward primer for amplification specific fragments of 16S rRNA gene in Nontuberculous mycobacteria
catgtcttctggtgcaaagctt

SEQ ID NO: 37 - DNA, 22 bp, Artificial Sequence
  forward primer for amplification specific fragments of 16S rRNA gene in Nontuberculous mycobacteria
catgttttctggtgcaaagctt

SEQ ID NO: 38 - DNA, 19 bp, Artificial Sequence
  forward primer(NTM-1) for amplification specific fragments of 16S rRNA gene in Nontuberculous mycobacteria
tktggtggaaagcttttgc

SEQ ID NO: 39 - DNA, 19 bp, Artificial Sequence
  forward primer(NTM-2) for amplification specific fragments of 16S rRNA gene in Nontuberculous mycobacteria
ggtgwgtggtgcaaagctt

SEQ ID NO: 40 - DNA, 18 bp, Artificial Sequence
  forward primer(NTM-3) for amplification specific fragments of 16S rRNA gene in Nontuberculous mycobacteria
tggtggaaagcgtttggt

SEQ ID NO: 41 - DNA, 19 bp, Artificial Sequence
  forward primer(NTM-1) for amplification specific fragments of 16S rRNA gene in Nontuberculous mycobacteria
tgtggtggaaagcttttgc

SEQ ID NO: 42 - DNA, 19 bp, Artificial Sequence
  forward primer(NTM-1) for amplification specific fragments of 16S rRNA gene in Nontuberculous mycobacteria
tttggtggaaagcttttgc

SEQ ID NO: 43 - DNA, 19 bp, Artificial Sequence
  forward primer(NTM-2) for amplification specific fragments of 16S rRNA gene in Nontuberculous mycobacteria
ggtgagtggtgcaaagctt

SEQ ID NO: 44 - DNA, 19 bp, Artificial Sequence
  forward primer(NTM-2) for amplification specific fragments of 16S rRNA gene in Nontuberculous mycobacteria
ggtgtgtggtgcaaagctt

(19) United States
(12) Patent Application Publication
(10) Pub. No.: US 2013/0164756 A1
(43) Pub. Date: Jun. 27, 2013

(54) METHOD FOR DETECTING MYCOBACTERIUM TUBERCULOSIS AND NONTUBERCULOUS MYCOBACTERIA USING DUPLEX REAL-TIME POLYMERASE CHAIN REACTION AND MELTING CURVE ANALYSIS

(71) Applicant: Jeong-Uk Kim, Gangneung-si (KR)

(72) Inventor: Jeong-Uk Kim, Gangneung-si (KR)

(73) Assignee: UNIVERSITY OF ULSAN FOUNDATION FOR INDUSTRY COOPERATION, Ulsan (KR)

(21) Appl. No.: 13/700,158

(22) PCT Filed: May 26, 2011
(86) PCT No.: PCT/KR2011/003858

§ 371 Date: Mar. 12, 2013

(30) Foreign Application Priority Data
May 27, 2010 (KR) 10-2010-0049691
Jun. 30, 2010 (KR) 10-2010-0062846
Jan. 31, 2011 (KR) 10-2011-0009529

(51) Int. Cl. C12Q 1/68

(52) U.S. Cl. CPC C12Q 1/689
USPC 435/6.12

(57) ABSTRACT

Disclosed are primer sets specific for the IS6110 gene characteristic of MTC and for the 16S rRNA gene characteristic of NTM, kits for the detection of MTC and NTM, comprising the same, and methods for detecting MTC and NTM by duplex real-time PCR and melting profile analysis using the same. Because the primers are exclusive to Mycobacterium tuberculosis and nontuberculous mycobacteria, the methods can be a clinical diagnostic means useful for the detection of both Mycobacterium tuberculosis and nontuberculous mycobacteria at the same time, with higher efficiency.

### TECHNICAL FIELD

The present invention relates to the detection of Mycobacterium tuberculosis and nontuberculous mycobacteria. More particularly, the present invention relates to a primer set capable of detecting specific nucleotide sequences of Mycobacterium tuberculosis and nontuberculous mycobacteria, a kit for the detection of Mycobacterium tuberculosis and nontuberculous mycobacteria, comprising the same, and a method for detecting Mycobacterium tuberculosis and nontuberculous mycobacteria simultaneously, by duplex real-time PCR and melting profile analysis using the same.

### BACKGROUND ART

Nontuberculous mycobacteria are widely distributed in the environment, particularly in wet soil, marshland and rivers, and had been recognized as non-pathogenic bacteria before the discovery of its opportunistic characteristics. In the 1980s, nontuberculous mycobacteria were found to be an opportunistic pathogen of pulmonary diseases in patients with acquired immunodeficiency syndrome (AIDS). Further, the bacteria were also known to cause diseases in other patients. With the report of the pathogenesis of nontuberculous mycobacteria, its clinical significance has been increasingly recognized.

Recently, the United States of America and many European countries, which have a low prevalence of tuberculosis, have seen an increase in the incidence of infections caused by nontuberculous mycobacteria. In Korea, the isolation of nontuberculous mycobacteria from clinical specimens has also increased, although the incidence of tuberculosis has been greatly reduced. Thanks to a policy granting medical insurance for the performing of liquid culture of tuberculosis bacteria in 2009 in Korea, there was an increase in the number of laboratories performing liquid culture. Liquid culture detects nontuberculous mycobacteria more often than does solid culture. Reports showed that nontuberculous mycobacteria was detected in about 12% of smear-positive tuberculosis cases as measured by liquid culture, with nontuberculous mycobacteria separated from the sputum accounting for about 10˜20% of pulmonary disease cases in Japan, Hong Kong, and Korea, and for about 40˜50% of pulmonary disease in the USA, Canada, and West Europe.

Resembling tuberculosis, which progresses slowly, the pulmonary diseases caused by nontuberculous mycobacteria are apt to be diagnosed wrongly. However, since drugs effective for the inhibition of Mycobacterium tuberculosis are different from those inhibitory of nontuberculous mycobacteria, there is a demand for rapid and accurate separation between tubercle bacilli and nontuberculous mycobacteria so that suitable drugs can be selected.

The conventional reagents in which the primer specific for the genus Mycobacterium is used as the detecting one for nontuberculous mycobacteria are problematic in terms of the accuracy. For example, when only a certain concentration of Mycobacterium tuberculosis is present, the reagents do not respond to primers for detecting Mycobacterium tuberculosis, but only to primers for nontuberculous mycobacteria, so Mycobacterium tuberculosis is wrongly identified as nontuberculous mycobacteria. On the other hand, when nontuberculous mycobacteria coexist with Mycobacterium tuberculosis, only nontuberculous mycobacteria are likely to be detected. Therefore, there is a need for a primer set capable of recognizing a nucleotide sequence that is absent from Mycobacterium tuberculosis, but intrinsic to nontuberculous mycobacteria, and for a method for separately detecting Mycobacterium tuberculosis and nontuberculous mycobacteria with rapidity and accuracy using the same.

### DISCLOSURE

### Technical Problem

It is an object of the present invention to provide a primer set specific for the IS6110 gene exclusive to Mycobacterium tuberculosis, and a primer set specific for the 16S rRNA gene of nontuberculous mycobacterium, both of which are applicable to the accurate detection and diagnosis of Mycobacterium tuberculosis and nontuberculous mycobacterium, separately.

It is another object of the present invention to provide a kit for the detection of Mycobacterium tuberculosis and nontuberculous mycobacterium, comprising a primer set specific for the IS6110 gene exclusive to Mycobacterium tuberculosis, and a primer set specific for the 16S rRNA gene of nontuberculous mycobacterium, both of which are applicable to the accurate detection and diagnosis of Mycobacterium tuberculosis and nontuberculous mycobacterium, separately

It is another object of the present invention to provide a method for the detection and diagnosis of Mycobacterium tuberculosis and nontuberculous mycobacteria with accuracy using a duplex real-time polymerase chain reaction based on the primer set, and a melting profile.

### Technical Solution

In accordance with an aspect thereof, the present invention provides a primer set specific for the 16S rRNA gene of nontuberculous mycobacteria, comprising a forward primer having the nucleotide sequence of SEQ ID NO: 3; at least one reverse primer selected from the group consisting of nucleotide sequences of SEQ ID NOS: 4 to 6; and a reverse primer having the nucleotide sequence of SEQ ID NO: 7.

In accordance with another aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7.

In accordance with a further aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7; heating the amplified DNA product over various temperature to produce a melting profile; and analyzing the melting profile to determine a melting temperature of the amplified product.

In accordance with still another aspect thereof, the present invention provides a primer set specific for the 16S rRNA gene of nontuberculous mycobacteria, comprising a forward primer having the nucleotide sequence of SEQ ID NO: 20 and a reverse primer having the nucleotide sequence of SEQ ID NO: 21.

In accordance with a still further aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer having the nucleotide sequence of SEQ ID NO: 20 and a reverse primer having the nucleotide sequence of SEQ ID NO: 21.

In accordance with yet another aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer having the nucleotide sequence of SEQ ID NO: 20 and a reverse primer having the nucleotide sequence of SEQ ID NO: 21; heating the amplified DNA product over various temperature to produce a melting profile; and analyzing the melting profile to determine a melting temperature of the amplified product.

In accordance with a yet further aspect thereof, the present invention provides a primer set specific for the 16S rRNA gene of nontuberculous mycobacteria, comprising a forward primer composed of respective primers having the nucleotide sequences of SEQ ID NOS: 38 to 40; and a reverse primer having the nucleotide sequence of SEQ ID NO: 21.

In accordance with still yet another aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer composed of respective primers having the nucleotide sequences of SEQ ID NOS: 38 to 40, and a reverse primer having the nucleotide sequence of SEQ ID NO: 21.

In accordance with a still yet further aspect thereof, the present invention provides a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer composed of respective primers having the nucleotide sequences of SEQ ID NOS: 38 to 40, and a reverse primer having the nucleotide sequence of SEQ ID NO: 21; heating the amplified DNA product over various temperature to produce a melting profile; and analyzing the melting profile to determine a melting temperature of the amplified product.

### Advantageous Effects

As described above, primer sets for the detection of Mycobacterium tuberculosis and nontuberculous mycobacteria, capable of detecting nucleotide sequences characteristic of Mycobacterium tuberculosis and nontuberculous mycobacteria, detection kits, and duplex real-time PCR-based methods for detecting Mycobacterium tuberculosis and nontuberculous mycobacteria using the primer sets, are provided by the present invention. Because no specific probes are employed, the method of the present invention can be a clinical diagnostic means useful for the simultaneous detection of both Mycobacterium tuberculosis and nontuberculous mycobacteria at low cost, with higher efficiency.

### DESCRIPTION OF DRAWINGS

FIG. 1 is a melting curve of an amplicon obtained by carrying out a duplex real-time PCR for MTC according to Example 1.

FIG. 2 is a melting curve of an amplicon obtained by carrying out a duplex real-time PCR for NTM according to Example 1.

FIG. 3 is a melting curve of an amplicon obtained by carrying out a duplex real-time PCR for MTC+NTM according to Example 1.

FIG. 4 is a melting curve of an amplicon obtained by carrying out a duplex real-time PCR for MTC according to Example 2.

FIG. 5 is a melting curve of an amplicon obtained by carrying out a duplex real-time PCR for NTM according to Example 2.

FIG. 6 is a melting curve of an amplicon obtained by carrying out a duplex real-time PCR for MTC+NTM according to Example 2

FIG. 7 is a melting curve of an amplicon obtained by carrying out a duplex real-time PCR for MTC according to Example 3.

FIG. 8 is a melting curve of an amplicon obtained by carrying out a duplex real-time PCR for NTM according to Example 3.

FIG. 9 is a melting curve of an amplicon obtained by carrying out a duplex real-time PCR for MTC+NTM according to Example 3.

### BEST MODE

In accordance with an aspect thereof, the present invention addresses a primer set specific for the 16S rRNA gene of nontuberculous mycobacteria, comprising a forward primer having the nucleotide sequence of SEQ ID NO: 3; at least one reverse primer selected from the group consisting of nucleotide sequences of SEQ ID NOS: 4 to 6; and a reverse primer having the nucleotide sequence of SEQ ID NO: 7.

The nucleotide sequence of SEQ ID NO 3 is a forward primer specific for the 16S rRNA of nontuberculous mycobacteria. The nucleotide sequence of SEQ ID NO: 3 (5′-ggyrayctgccctgcac-3′) refers to a primer set comprising 5′-ggtaatctgccctgcac-3′ (SEQ ID NO: 8), 5′-ggtaacctgccctgcac-3′ (SEQ ID NO: 9), 5′-ggcaatctgccctgcac-3′ (SEQ ID NO: 10), 5′-ggcaacctgccctgcac-3′ (SEQ ID NO: 11), 5′-ggtgatctgccctgcac-3′ (SEQ ID NO: 12), 5′-ggtgacctgccctgcac-3′ (SEQ ID NO: 13), 5′-ggcgatctgccctgcac-3′ (SEQ ID NO: 14), and 5′-ggcgacctgccctgcac-3′ (SEQ ID NO: 15). For example, it may be a primer set in which 5′-ggtaatctgccctgcac-3′, 5′-ggtaacctgccctgcac-3′, 5′-ggcaatctgccctgcac-3′, 5′-ggcaacctgccctgcac-3′, 5′-ggtgatctgccctgcac-3′, 5′-ggtgacctgccctgcac-3′, 5′-ggcgatctgccctgcac-3′, and 5′-ggcgacctgccctgcac-3′ are mixed at a ratio of approximately 1:1:1:1:1:1:1:1.

All of the nucleotide sequences of SEQ ID NOS: 4, 5 and 6 (NTM-1) are reverse primers specific for the 16S rRNA. The nucleotide sequence of SEQ ID NO: 7 (NTM-2) is also a reverse primer specific for the 16S rRNA gene of nontuberculous mycobacteria. All of the reverse primers for the 16S rRNA gene of nontuberculous mycobacteria are designed to detect all of various target nontuberculous mycobacterium species. The reverse primer of SEQ ID NO: 7 (5′-catcccacaccgctaccw-3′) refers to a primer set comprising 5′-catcccacaccgctacct-3′ (SEQ ID NO: 16), and 5′-catcccacaccgctacca-3′ (SEQ ID NO: 17). For example, the nucleotide sequence of SEQ ID NO: 7 may be a primer set in which 5′-catcccacaccgctacct-3′ 5′-catcccacaccgctacca-3′ are mixed at a ratio of approximately 1:1.

In accordance with another aspect thereof, the present invention addresses a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7.

The kit for the detection of Mycobacterium tuberculosis and nontuberculous mycobacteria comprises a primer set, specific for the IS6110 of Mycobacterium tuberculosis, which consists of a forward primer and a reverse primer having the nucleotide sequences of SEQ ID NOS: 1 and 2, respectively. The primer set specific for the IS6100 gene is designed to detect various mycobacterium species (Mycobacterium tuberculosis complex, MTC).

The kit for the detection of Mycobacterium tuberculosis and nontuberculous mycobacteria comprises a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6 (NTM-1), and a reverse primer having the nucleotide sequence of SEQ ID NO: 7 (NTM-2). The reverse primers for the 16S rRNA gene of nontuberculous mycobacteria are designed to detect various nontuberculous mycobacterium species.

In one embodiment, the kit for the detection of Mycobacterium tuberculosis and nontuberculous mycobacteria may comprise the reverse primers having the nucleotide sequences of SEQ ID NOS: 4 and 7 at a ratio of 1:1, the latter being composed of SEQ ID NOS: 16 and 17 at a ratio of 1:1. Accordingly, in the kit, the primer of SEQ ID NO:4, 5′-catcccacaccgctacct-3′ (SEQ ID NO: 16), and 5′-catcccacaccgctacca-3′ (SEQ ID NO: 17) may be mixed at a ratio of 2:1:1.

In another embodiment of the present invention, the kit for the detection of Mycobacterium tuberculosis and nontuberculous mycobacteria may comprise the reverse primers having the nucleotide sequences of SEQ ID NOS: 5 and 7 at a ratio of 1:1, the latter being composed of SEQ ID NOS: 16 and 17 at a ratio of 1:1.

In another embodiment of the present invention, the kit for the detection of Mycobacterium tuberculosis and nontuberculous mycobacteria may comprise the reverse primers having the nucleotide sequences of SEQ ID NOS: 6 and 7 at a ratio of 1:1, the latter being composed of SEQ ID NOS: 16 and 17 at a ratio of 1:1.

The detection kit may further comprise a reagent necessary for amplifying DNA by PCR. This reagent may include DNA polymerase, dNTPs, PCR buffer, etc. For use in the kit for the detection of Mycobacterium tuberculosis and nontuberculous mycobacteria, primers are capable of detecting genes characteristic of Mycobacterium tuberculosis and nontuberculous mycobacteria and may be designed to allow for the amplification of PCR products which are greatly different in melting temperature. Because the melting profile of the PCR product varies depending on various factors including DNA size and GC ratio, these factors may be taken into account when the primer sets are designed.

According to one embodiment of the present invention, the kit for detection of Mycobacterium tuberculosis and nontuberculous mycobacteria may further comprise a fluorescent dye, such as SYT09, EvaGreen or LCGreen, but does not contain SYBR Green because this fluorescent dye not only inhibits the polymerase chain reaction, when it is used in a high concentration, but also is redistributed in the course of melting of the product, exerting negative effects on the analysis of the high-resolution melting curve which may allow for the determination of genotype. That is, a reaction mixture for use in PCR and melting profile analysis may contain a fluorescent dye such as SYTO 9, EvaGreen, or LCGreen. For example, a reaction mixture containing EvaGreen may be used for PCR and melting profile analysis. In one embodiment, real-time PCR was performed with the aid of a Type-it HRM PCR kit (QIAGEN Inc., Germantown, Md., USA). This kit contains EvaGreen.

In accordance with a further aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7; heating the amplified DNA product over various temperature to produce a melting profile; and analyzing the melting profile to determine a melting temperature of the amplified product.

In one embodiment of the present invention, the analysis of the melting profile may be performed by measuring absorbance at 510 nm.

In accordance with still another aspect thereof, the present invention addresses a primer set specific for the 16S rRNA gene of nontuberculous mycobacteria, comprising a forward primer having the nucleotide sequence of SEQ ID NO: 20 and a reverse primer having the nucleotide sequence of SEQ ID NO: 21.

The nucleotide sequence of SEQ ID NO 20 is a forward primer specific for the 16S rRNA of nontuberculous mycobacteria. The forward primer for the 16S rRNA gene of nontuberculous mycobacteria is designed to detect various nontuberculous mycobacterium species. The nucleotide sequence of SEQ ID NO: 20 refers to a primer set comprising 5′-catgtcttgtgggggaaagctt-3′ (SEQ ID NO: 22), 5′-catgttttgtgggggaaagctt-3′ (SEQ ID NO: 23), 5′-catgtcttctgggggaaagctt-3′ (SEQ ID NO: 24), 5′-catgtcttgtggtggaaagctt-3′ (SEQ ID NO: 25), 5′-catgtcttgtggggcaaagctt-3′ (SEQ ID NO: 26), 5′-catgttttctgggggaaagctt-3′ (SEQ ID NO: 27), 5′-catgtcttctggtggaaagctt-3′ (SEQ ID NO: 28), 5′-catgtcttgtggtgcaaagctt-3′ (SEQ ID NO: 29), 5′-catgttttgtggggcaaagctt-3′ (SEQ ID NO: 30), 5′-catgtcttctggggcaaagctt-3′ (SEQ ID NO: 31), 5′-catgttttgtggtggaaagctt-3′ (SEQ ID NO: 32), 5′-catgttttctggtggaaagctt-3′ (SEQ ID NO: 33), 5′-catgttttctggggcaaagctt-3′ (SEQ ID NO: 34), 5′-catgttttgtggtgcaaagctt-3′ (SEQ ID NO: 35), 5′-catgtcttctggtgcaaagctt-3′ (SEQ ID NO: 36), 5′-catgttttctggtgcaaagctt-3′ (SEQ ID NO: 37) at a ratio of approximately 1:1:1:1:1:1:1:1:1:1:1:1:1:1:1:1.

The nucleotide sequence of SEQ ID NO: 21 is used as a reverse primer specific for the 16S rRNA of nontuberculous mycobacteria.

In accordance with a still further aspect thereof, the present invention addresses a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer having the nucleotide sequence of SEQ ID NO: 20 and a reverse primer having the nucleotide sequence of SEQ ID NO: 21.

In the kit for the detection of Mycobacterium tuberculosis and nontuberculous mycobacteria, the primer set specific for the IS6110 gene of Mycobacterium tuberculosis, composed of a forward primer having the nucleotide sequence of SEQ ID NO: 18 and a reverse primer having the nucleotide sequence of SEQ ID NO: 19, is designed to detect various Mycobacterium species (Mycobacterium tuberculosis complex, MTC).

In the kit for the detection of Mycobacterium tuberculosis and nontuberculous mycobacteria, the primer set specific for the IS6110 of Mycobacterium tuberculosis, composed of a forward primer and a reverse primer having the nucleotide sequences of SEQ ID NOS: 20 and 21, respectively, is designed to detect various nontuberculous mycobacteria.

The detection kit may further comprise a reagent necessary for amplifying DNA by PCR. This reagent may include DNA polymerase, dNTPs, PCR buffer, etc. For use in the kit for the detection of Mycobacterium tuberculosis and nontuberculous mycobacteria, primers are capable of detecting genes characteristic of Mycobacterium tuberculosis and nontuberculous mycobacteria and may be designed to allow for the amplification of PCR products which are greatly different in melting temperature. Because the melting profile of the PCR product varies depending on various factors including DNA size and GC ratio, these factors may be taken into account when the primer sets are designed.

According to one embodiment of the present invention, the kit for detection of Mycobacterium tuberculosis and nontuberculous mycobacteria may further comprise a fluorescent dye, such as SYTO9, EvaGreen or LCGreen, but does not contain SYBR Green because this fluorescent dye not only inhibits the polymerase chain reaction, when it is used in a high concentration, but also is redistributed in the course of melting of the product, exerting negative effects on the analysis of the high-resolution melting curve which may allow for the determination of genotype. That is, a reaction mixture for use in PCR and melting profile analysis may contain a fluorescent dye such as SYTO 9, EvaGreen, or LCGreen. For example, a reaction mixture containing EvaGreen may be used for PCR and melting profile analysis. In one embodiment, real-time PCR was performed with the aid of a Type-it HRM PCR kit (QIAGEN Inc., Germantown, Md., USA). This kit contains EvaGreen.

In accordance with yet another aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer having the nucleotide sequence of SEQ ID NO: 20 and a reverse primer having the nucleotide sequence of SEQ ID NO: 21; heating the amplified DNA product over various temperature to produce a melting profile; and analyzing the melting profile to determine a melting temperature of the amplified product.

In accordance with a yet further aspect thereof, the present invention addresses a primer set specific for the 16S rRNA gene of nontuberculous mycobacteria, comprising a forward primer composed of respective primers having the nucleotide sequences of SEQ ID NOS: 38 to 40; and a reverse primer having the nucleotide sequence of SEQ ID NO: 21.

All of the nucleotide sequences of SEQ ID NOS: 38, 39, and (NTM-1) are forward primers specific for the 16S rRNA of nontuberculous mycobacteria. The forwards primers specific for 16S rRNA of nontuberculous mycobacteria are designed to detect various nontuberculous mycobacteria.

The forward primers may form a primer set in which 5′-tgtggtggaaagcttttgc-3′ (SEQ ID NO: 41), and 5′-tttggtggaaagcttttgc-3′ (SEQ ID NO: 42) are mixed at a ratio of approximately 1:1.

Alternatively, the forward primers may form a primer set comprising 5′-ggtgagtggtgcaaagctt-3′ (SEQ ID NO: 43), and 5′-ggtgtgtggtgcaaagctt-3′ (SEQ ID NO: 44) at a ratio of approximately 1:1.

The nucleotide sequence of SEQ ID NO: 21 is used as a reverse primer specific for the 16S rRNA gene of nontuberculous mycobacteria.

In accordance with still yet another aspect thereof, the present invention addresses a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer composed of respective primers having the nucleotide sequences of SEQ ID NOS: 38 to 40, and a reverse primer having the nucleotide sequence of SEQ ID NO: 21.

In the kit for the detection of Mycobacterium tuberculosis and nontuberculous mycobacteria, the primer set specific for the IS6110 gene of Mycobacterium tuberculosis, composed of a forward primer having the nucleotide sequence of SEQ ID NO: 18 and a reverse primer having the nucleotide sequence of SEQ ID NO: 19, are designed to detect various mycobacterium species (Mycobacterium tuberculosis complex, MTC).

In the kit for the detection of Mycobacterium tuberculosis and nontuberculous mycobacteria, the primer set specific for the 16S rRNA gene of nontuberculous mycobacteria, comprising a forward primer composed of respective primers having the nucleotide sequences of SEQ ID NOS: 38 to 40, and a reverse primer having the nucleotide sequence of SEQ ID NO: 21, are designed to detect various nontuberculous mycobacteria.

The detection kit may further comprise a reagent necessary for amplifying DNA by PCR. This reagent may include DNA polymerase, dNTPs, PCR buffer, etc. For use in the kit for the detection of Mycobacterium tuberculosis and nontuberculous mycobacteria, primers are capable of detecting genes characteristic of Mycobacterium tuberculosis and nontuberculous mycobacteria and may be designed to allow for the amplification of PCR products which are greatly different in melting temperature. Because the melting profile of the PCR product varies depending on various factors including DNA size and GC ratio, these factors may be taken into account when the primer sets are designed.

According to one embodiment of the present invention, the kit for detection of Mycobacterium tuberculosis and nontuberculous mycobacteria may further comprise a fluorescent dye, such as SYTO 9, EvaGreen or LCGreen, but does not contain SYBR Green because this fluorescent dye not only inhibits the polymerase chain reaction, when it is used in a high concentration, but also is redistributed in the course of melting of the product, exerting negative effects on the analysis of the high-resolution melting curve which may allow for the determination of genotype. That is, a reaction mixture for use in PCR and melting profile analysis may contain a fluorescent dye such as SYTO 9, EvaGreen, or LCGreen. For example, a reaction mixture containing EvaGreen may be used for PCR and melting profile analysis. In one embodiment, real-time PCR was performed with the aid of a Type-it HRM PCR kit (QIAGEN Inc., Germantown, Md., USA). This kit contains EvaGreen.

In accordance with a still yet further aspect thereof, the present invention addresses a method for detecting Mycobacterium tuberculosis and nontuberculous mycobacteria, comprising: isolating DNA from a test subject; amplifying the DNA by duplex real-time PCR using a primer set specific for the IS6110 gene of Mycobacterium tuberculosis, composed of a forward primer having the nucleotide sequence of SEQ ID NO: 18 and a reverse primer having the nucleotide sequence of SEQ ID NO: 19; and a primer set specific for the 16S rRNA gene of nontuberculous mycobacteria, comprising a forward primer composed of respective primers having the nucleotide sequences of SEQ ID NOS: 38 to 40, and a reverse primer having the nucleotide sequence of SEQ ID NO: 21; heating the amplified DNA product over various temperature to produce a melting profile; and analyzing the melting profile to determine a melting temperature of the amplified product.

In accordance with an embodiment of the present invention, the melting profile analysis may be carried out by measuring absorbance at 510 nm.

### MODE FOR INVENTION

A better understanding of the present invention may be obtained through the following examples which are set forth to illustrate, but are not to be construed as limiting the present invention.

### Reference Example

### Search for Nucleotide Sequence Characteristic of Mycobacterium tuberculosis and Nontuberculous Mycobacteria

1. Target and Gene Loci Used in searching for nucleotide sequences characteristic of Mycobacterium tuberculosis and nontuberculous mycobacteria were data of 16S ribosomal RNA genes of the following mycobacteria:

M. abscessus (AJ419970.1, AJ416940.1, AJ536038), M. acapulcensis (AF480575.1), M. africanum (AF480605.1), M. agri (AJ429045.1), M. aichiense (X55598.1), M. alvei (NR_{—}024859.1), M. asiaticum (X55604.1), M. aurum (FJ172298.1), M. austroafricanum (GU121552.1), M. avium (NR_{—}025584.1, AJ536037.1, EF521892.1), M. bohemicum (NR_{—}026054.1), M. botniense (NR_{—}028878.1), M. bovis (GU142937.1), M. branderi (AF480574.1), M. brumae (NR_{—}025233.1), M. celatum (L08169.1), M. chelonae (AM884324.1, AJ419969.1), M. chitae (NR_{—}029220.1), M. chlorophenolicum (NR_{—}026173.1), M. chubuense (X55596.1), M. confluentis (AJ634379.1), M. conspicuum (X029298.1), M. cookii (X53896.1), M. diernhoferi (AF480599.1), M. doricum (NR_{—}025099.1), M. duvalii (NR_{—}026073.1), M. engbaekii (AF480577.1), M. fallax (AF480600.1), M. farcinogenes (X55592.1), M. flavescens (AY734993.1), M. fortuitum (AY457066.1, AF480580.1, GU142933.1), M. gadium (NR_{—}026087.1), M. gastri (GU142918.1), M. genavense (NR_{—}029223.1), M. gilvum (AB491971.1), M. goodii (AY457079.1), M. gordonae (GU142923.1), M. haemophilum (V06638.1), M. hassiacum (NR_{—}026011.1), M. heidelbergense (NR_{—}025268.1), M. hiberniae (NR_{—}026092.1), M. hodleri (NR_{—}026286.1), M. immunogen (AJ011771.1), M. interjectum (X70961.1), M. intermedium (X67847.1), M. intracellulare (AY652958.1, AJ536036.1, X52927.1, M61684.1), M. kansasii (M29575.1, X15916.1), M. lentiflavum (AF480583.1), M. mageritense (AY457076.1), M. malmoense (GQ153278.1), M. marinum (AF456238.1, AY513243.1), M. microti (NR_{—}025234.1), M. monacense (GU142931.1), M. moriokaense (AY859686.1), M. mucogenicum (AF480585.1), M. neoaurum (FJ172306.1), M. nonchromogenicum (DQ058406.1), M. obuense (X55597.1), M. paraffinicum (GQ153282.1), M. parafortuitum (NR_{—}026285.1), M. peregrinum (AY457069.1), M. phlei (AF480603.1), M. porcinum (AY457077.1), M. poriferae (NR_{—}025235.1), M. pulveris (NR_{—}025528.1), M. rhodesiae (NR_{—}025529.1), M. scrofulaceum (GQ153271.1), M. senuense (DQ536409.1), M. septicum (AY457070.1), M. shimoidei (X82459.1), M. simiae (GQ153280.1), M. smegmatis (NR_{—}025311.1), M. sphagni (X55590.1), M. szulgai (X52926.1), M. terrae (NR_{—}029168.1), M. thermoresistibile (GU142928.1), M. tilburgii (AJ580826.1), M. triplex (GQ153279.1), M. triviale (DQ058405.1), M. tuberculosis (GU142936.1, GU142935.1, AY53603.1, X55588.1, X52917.1), M. tusciae (NR_{—}024903.1), M. ulcerans (Z13990.1), M. vaccae (X55601.1), M. wolinskyi (AY457083.1), and M. xenopi (X52929.1). The data of the 16S ribosomal RNA genes were obtained from the database of the NCBI (National center for Biotechnology Information).

Analysis of the data of 16S rRNA gene sequences of the mycobacterium species with the aid of Sequencher 4.9 resulted in characteristic nucleotide sequences, that is, nucleotides characteristic of Mycobacterium tuberculosis complex, and nucleotides absent from Mycobacterium tuberculosis complex, but intrinsic to nontuberculous mycobacteria.

### Example 1

### Separation and Detection of Mycobacterium tuberculosis Complex and Nontuberculous Mycobacteria 1

1. Detection Target and Primer Design

Target genes to be detected by PCR and melting profile analysis were the IS6110 gene for Mycobacterium tuberculosis complex (M. tuberculosis, M. bovis, M. africanum, M. microti), and the 16S rRNA gene for nontuberculous mycobacteria. A universal primer was used as a forward primer to amplify the 16S rRNA gene of mycobacteria. NTM-1 and NTM-2, which are characteristic of nontuberculous mycobacteria, were used as reverse primers. These primers useful in the detection of target genes were designed using the Primer3 program.

(1) Mycobacterium tuberculosis Complex (MTC)

1) target gene: IS6110

2) primers

a. forward primer:
(SEQ ID NO: 1)
5′-cgaactcaaggagcacatca-3′
b. reverse primer:
(SEQ ID NO: 2)
5′-agtttggtcatcagccgttc-3′

3) PCR product size: 135 bp

4) mean melting temperature of PCR product (Tm): about 86.18±0.16° C. (Tm slightly differed from one batch to another)

(2) Nontuberculous Mycobacteria (NTM)

1) target gene: 16S rRNA

2) primers

a. forward primer:
(SEQ ID NO: 3)
5′-ggyrayctgccctgcac-3′
b. reverse primer NTM-1:
(SEQ ID NO: 4)
5′-cccacaccgcaaaagctt-3′,
(SEQ ID NO: 5)
5′-cccacaccgcaaaagct-3′
or
(SEQ ID NO: 6)
5′-tcccacaccgcaaaagct-3′
NTM-2:
(SEQ ID NO: 7)
5′-catcccacaccgctaccw-3′

3) PCR product size: 104 bp

4) mean melting temperature of PCR product (Tm): about 80.10° C. to 82.10° C. (depending on species of nontuberculous mycobacteria, Tm varied, but within a range of from about 80° C. to 82° C.).

### Example 1-1

### Duplex Real-Time PCR Using the Nucleotide Sequence of SEQ ID NO: 4 as a Reverse Primer NTM-1 for the Detection of NTM, and Melting Profile Analysis

(1) Isolation of DNA

DNA was isolated from 186 Mycobacterium species and 78 nontuberculous mycobacterium species, all recovered from clinical specimens, and from 7 standard ATCC mycobacteria species including M. tuberculosis (ATCC 25177), M. intracellulare (ATCC 13950), M. scrofulaceum (ATCC 19981), M. kansasii (ATCC 12478), M. fortuitum (ATCC 6841), M. abscessus (ATCC 19977), and M. avium (ATCC 25291).

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC standard species were cultured in broths.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex PCR and Melting Profile Analysis

Duplex real-time PCR started with denaturation at 95° C. for 5 min and was run with 40 cycles of denaturation at 95° C. for 10 sec, and annealing at 60° C. for 30 sec, and extension, using a Type-it HRM PCR kit (QIAGEN Inc., Germantown, Md., USA). This kit contained the fluorescent EvaGreen. After completion of the PCR, melting profile analysis was carried out by measuring absorbance at 510 nm while the PCR product was heated from 77° C. to 91.5° C. at an elevation rate of 0.2° C./sec. Because PCR depends on various factors including the concentrations of primers, the temperature and time of annealing and extension steps, the PCR conditions optimal for detecting both the target genes were determined in consideration of the factors. The composition of the PCR reagent is summarized in Table 1, below.

In the primer mix, a forward primer and a reverse primer were contained in the same amounts (10 pmoles/μL). Accordingly, 1.75 μL of the primer mix for MTC contained the forward primer and the reverse primer in an amount of 17.5 pmoles, each. Since a total volume of PCR mixture was 25 μL, it contained the primers at a concentration of 0.7 μM (17.5 pmoles/25 μL). For NTM, each of the forward primer and the reverse primer was in an amount of 10 pmoles/1 μL, with a final concentration of 0.4 μM (10 pmoles/25 μL). In this context, the NTM forward primer was a set of 5′-ggtaatctgccctgcac-3′ (SEQ ID NO: 8), 5′-ggtaacctgccctgcac-3′ (SEQ ID NO: 9), 5′-ggcaatctgccctgcac-3′ (SEQ ID NO: 10), 5′-ggcaacctgccctgcac-3′ (SEQ ID NO: 11), 5′-ggtgatctgccctgcac-3′ (SEQ ID NO: 12), 5′-ggtgacctgccctgcac-3′ (SEQ ID NO: 13), 5′-ggcgatctgccctgcac-3′ (SEQ ID NO: 14), and 5′-ggcgacctgccctgcac-3′ (SEQ ID NO: 15). In the primer set, the primers of SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15 were used at a ratio of about 1:1:1:1:1:1:1:1. That is, the primer set contained 1.25 pmoles of each of the primers of SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15. On the other hand, each of the NTM-1 reverse primer (SEQ ID NO: 4) and the NTM-2 reverse primer (SEQ ID NO: 7) were used in an amount of 10 pmoles while the NTM-2 reverse primer was comprised of 5 pmoles of 5′-catcccacaccgctacct-3′ (SEQ ID NO: 16) and 5 pmoles of 5′-catcccacaccgctacca-3′ (SEQ ID NO: 17).

TABLE 1

| Ingredient | | Vol. (μL) | Conc. |
|---|---|---|---|
| 2X HRM PCR Master Mix | | 12.5 | 1X |
| Primer Mix (10 pmole/μL) | MTC | 1.75 | 0.7 uM |
| Primer Mix (10 pmole/μL) | NTM | 1.0 | 0.4 uM |

| | | |
|---|---|---|
| Nuclease free water | 4.75 | — |
| Sample DNA template | 5 | — |
| Total | 25 | — |

### Example 1-2

### Duplex Real-Time PCR Using the Nucleotide Sequence of SEQ ID NO: 5 as the Reverse Primer NTM-1 for the Detection of NTM, and Melting Profile Analysis

Duplex real-time PCR and melting profile analysis were carried out in the same manner as in Example 1-1, with the exception that the nucleotide sequence of SEQ ID NO: 5 was used as the reverse primer NTM-1.

### Example 1-3

### Duplex Real-Time PCR Using the Nucleotide Sequence of SEQ ID NO: 6 as the Reverse Primer NTM-1 for the Detection of NTM, and Melting Profile Analysis

Duplex real-time PCR and melting profile analysis were carried out in the same manner as in Example 1-1, with the exception that the nucleotide sequence of SEQ ID NO: 6 was used as the reverse primer NTM-1.

2. Result of Duplex Real-Time PCR and Melting Profile Analysis

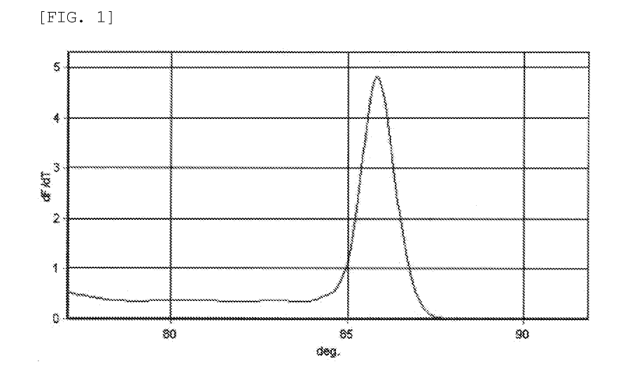

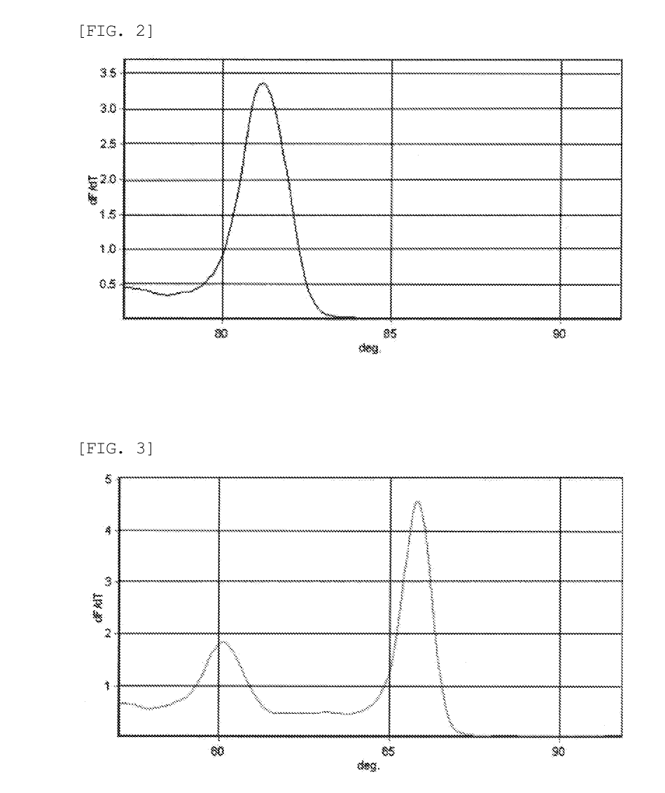

FIGS. 1 to 3 are melting profiles of the duplex real-time PCR products (amplicons) amplified from DNAs of the bacteria, that is, MTC, NTM, and MTC+NTM, respectively. In the profiles, temperatures (° C.) are set on the X-axis while a change in fluorescence intensity (F) per unit time (dF/dT) is on the Y-axis.

The MTC amplicon was measured to have a mean melting temperature (Tm) of about 86.18±0.16° C. while the NTM amplicon ranged in Tm from about 80.10° C. to 82.10° C. For MTC+NTM, two respective Tm peaks were read.

Therefore, the duplex real-time PCR and melting profile analysis according to the present invention was found to guarantee the detection of MTC and NTM from clinical subjects, simultaneously, with high reliability.

### Example 2

### Separation and Detection of Mycobacterium tuberculosis Complex and Nontuberculous Mycobacteria

1. Detection Target and Primer Design

Target genes to be detected by PCR and melting profile analysis were the IS6110 gene for Mycobacterium tuberculosis complex (M. tuberculosis, M. bovis, M. africanum, M. microti), and the 16S rRNA gene for nontuberculous mycobacteria. For use in the detection of nontuberculous mycobacteria, a forward primer was specific for the target gene characteristic of nontuberculous mycobacteria while a universal primer was used as a reverse primer. These primers useful in the detection of target genes were designed using the Primer3 program.

(1) Mycobacterium tuberculosis Complex (MTC)

1) target gene: IS6110

2) primers

a. forward primer:
(SEQ ID NO: 18)
5′-agaaggcgtactcgacctga-3′
b. reverse primer:
(SEQ ID NO: 19)
5′-ctgaaccggatcgatgtgta-3′

3) PCR product size: 75 bp

4) mean melting temperature of PCR product (Tm): 78.0±0.20° C.

(2) Nontuberculous Mycobacteria (NTM)

1) target gene: 16S rRNA

2) primers

a. forward primer:
(SEQ ID NO: 20)
5′-catgtyttstggkgsaaagctt-3′
b. reverse primer:
(SEQ ID NO: 21)
5′-cgtaggagtctgggccgta-3′

3) PCR product size: 152 bp

4) mean melting temperature of PCR product (Tm): about 86.7° C. to 88.24° C. (depending on species of nontuberculous mycobacteria, the Tm varied, but within a range of from about 86.5° C. to 88.5° C.)

2. Duplex PCR and Melting Profile Analysis

(1) Isolation of DNA DNA was isolated from 186 Mycobacterium species and 78 nontuberculous mycobacterium species, all recovered from clinical specimens, and from 68 standard ATCC mycobacteria species including M. abscessus ATCC 19977, M. acapulcensis KCTC 9501, M. africanum ATCC 25420, M. agri KCTC 9502, M. alvei KCTC 19709, M. asiaticum KCTC 9503, M. aurum KCTC 19457, M. austroafricanum KCTC 9504, M. avium ATCC 25291, M. bolletii KCTC 19281, M. botniense KCTC 19646, M. bovis ATCC 19210, M. brumae KCTC 19711, M. celatum ATCC 51131, M. chelonae subsp chelonae KCTC 9505, M. chlorophenolicum KCTC 19089, M. chubuense KCTC 19712, M. diernhoferi KCTC 9506, M. fallax KCTC 9508, M. flavescens ATCC 14474, M. fortuitum ATCC 6841, M. frederiksbergense KCTC 19100, M. gadium ATCC 27726, M. gastri ATCC 15754, M. gilvum KCTC 19423, M. goodii ATCC BAA-955, M. gordonae KCTC 9513, M. haemophilum ATCC 29548, M. hassiacum ATCC 700660, M. interjectum ATCC 51457, M. intermedium ATCC 51848, M. intracellulare ATCC 13950, M. intracellulare KCTC 9514, M. kansasii ATCC 12478, M. lentiflavum KMRC 70087, M. malmoense ATCC 29571, M. mantobense KCTC 9977, M. marinum ATCC 927, M. massiliense KCTC 19086, M. microti ATCC 19422, M. moriokaense KCTC 9516, M. mucogenicum KCTC 19088, M. neoaurum KCTC 19096, M. nonchromogenicum ATCC 19530, M. obuense KCTC 19097, M. parascrofulaceum KCTC 9979, M. peregrinum KCTC 9615, KMRC 75002, M. phlei KCTC 9689, M. porcinum KCTC 9517, M. pulveris KCTC 9518, M. scrofulaceum ATCC 19981, M. septicum ATCC 700731, M. simiae ATCC 25275, M. shimoidei ATCC 27962, M. smegmatis KCTC 9108, M. szulgai KCTC 9520, KMRC 31125, M. terrae KCTC 9614, M. triplex ATCC 700071, M. triviale KMRC 70093, M. tuberculosis ATCC 25177, ATCC 27294, M. ulcerans ATCC 19423, M. vaccae KCTC 19087, M. vanbaalenii KCTC 9966, M. wolinskyi ATCC 700010, and M. xenopi KMRC 42001.

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC and KCTC standard species were cultured in broths while the KMRC species was grown on an agar plate.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex PCR and Melting Profile Analysis

Duplex real-time PCR started with denaturation at 95° C. for 5 min and was run with 40 cycles of denaturation at 95° C. for 10 sec, annealing at 50° C. for 10 sec, and extension at 63° C. for 5 sec, using a Type-it HRM PCR kit (QIAGEN Inc., Germantown, Md., USA). This kit contained the fluorescent EvaGreen. After completion of the PCR, melting profile analysis was carried out by measuring absorbance at 510 nm while the PCR product was heated from 75° C. to 93.5° C. at an elevation rate of 0.2° C./sec. Because PCR depends on various factors including the concentrations of primers, the temperature and time of annealing and extension steps, the PCR conditions optimal for detecting both the target genes were determined in consideration of the factors. The composition of the duplex real-time PCR reagent is summarized in Table 2, below.

In the primer mix, a forward primer and a reverse primer were contained in the same amounts (10 pmoles/μL). Accordingly, 1.25 μL of the primer mix for MTC contained the forward primer and the reverse primer in an amount of 12.5 pmoles, each. Since a total volume of PCR mixture was 25 μL, it contained the primers at a concentration of 0.5 μM (12.5 pmoles/25 μL). For NTM, each of the forward primer and the reverse primer was in an amount of 15 pmoles/1.5 μL, with a final concentration of 0.6 μM (15 pmoles/25 μL). In this context, the NTM forward primer was a set of 5′-catgtcttgtgggggaaagctt-3′ (SEQ ID NO: 22), 5′-catgttttgtgggggaaagctt-3′ (SEQ ID NO: 23), 5′-catgtcttctgggggaaagctt-3′ (SEQ ID NO: 24), 5′-catgtcttgtggtggaaagctt-3′ (SEQ ID NO: 25), 5′-catgtcttgtggggcaaagctt-3′ (SEQ ID NO: 26), 5′-catgttttctgggggaaagctt-3′ (SEQ ID NO: 27), 5′-catgtcttctggtggaaagctt-3′ (SEQ ID NO: 28), 5′-catgtcttgtggtgcaaagctt-3′ (SEQ ID NO: 29), 5′-catgttttgtggggcaaagctt-3′ (SEQ ID NO: 30), 5′-catgtcttctggggcaaagctt-3′ (SEQ ID NO: 31), 5′-catgttttgtggtggaaagctt-3′ (SEQ ID NO: 32), 5′-catgttttctggtggaaagctt-3′ (SEQ ID NO: 33), 5′-catgttttctggggcaaagctt-3′ (SEQ ID NO: 34), 5′-catgttttgtggtgcaaagctt-3′ (SEQ ID NO: 35), 5′-catgtcttctggtgcaaagctt-3′ (SEQ ID NO: 36), and 5′-catgttttctggtgcaaagctt-3′ (SEQ ID NO: 37) which were mixed at a ratio of about 1:1:1:1:1:1:1:1:1:1:1:1:1:1:1:1.

TABLE 2

| Ingredient | | Vol. (μL) | Conc. |
|---|---|---|---|
| 2X HRM PCR Master Mix | | 12.5 | 1X |
| Primer Mix (10 pmole/μL) | MTC | 1.25 | 0.5 uM |
| Primer Mix (10 pmole/μL) | NTM | 1.5 | 0.6 uM |

| | | |
|---|---|---|
| Nuclease free water | 4.75 | — |
| Sample DNA template | 5 | — |
| Total | 25 | — |

3. Result of Duplex Real-Time PCR and Melting Profile Analysis

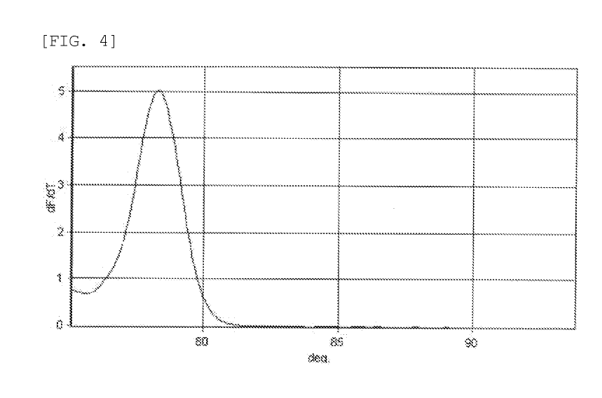

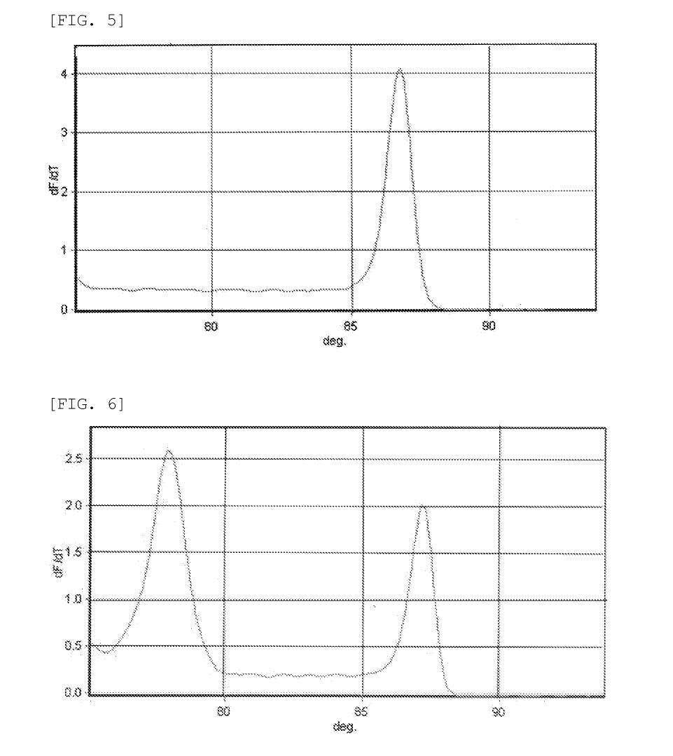

FIGS. 4 to 6 are melting profiles of the duplex real-time PCR products (amplicons) amplified from DNAs of the bacteria, that is, MTC, NTM, and MTC+NTM, respectively. In the profiles, temperatures (° C.) are set on the X-axis while a change in fluorescence intensity (F) per unit time (dF/dT) is on the Y-axis.

The MTC amplicon was measured to have a mean melting temperature (Tm) of about 78.0±0.20° C. while the NTM amplicon ranged in Tm from about 86.5° C. to 88.5° C. For MTC+NTM, two respective Tm peaks were read.

Therefore, the duplex real-time PCR and melting profile analysis according to the present invention was found to guarantee the detection of MTC and NTM from clinical subjects, simultaneously, with high reliability.

### Example 3

### Separation and Detection of Mycobacterium tuberculosis Complex and Nontuberculous Mycobacteria 3

1. Detection Target and Primer Design

Target genes to be detected by PCR and melting profile analysis were the IS6110 gene for Mycobacterium tuberculosis complex (M. tuberculosis, M. bovis, M. africanum, M. microti), and the 16S rRNA gene for nontuberculous mycobacteria. For use in the detection of nontuberculous mycobacteria, a forward primer was specific for the target gene characteristic of nontuberculous mycobacteria while a universal primer was used as a reverse primer. These primers useful in the detection of target genes were designed using the Primer3 program.

(1) Mycobacterium tuberculosis Complex (MTC)

1) target gene: IS6110

2) primers

a. forward primer:
(SEQ ID NO: 18)
5′-agaaggcgtactcgacctga-3′
b. reverse primer:
(SEQ ID NO: 19)
5′-ctgaaccggatcgatgtgta-3′

3) PCR product size: 75 bp

4) mean melting temperature of PCR product (Tm): 78.0±0.20° C.

(2) Nontuberculous Mycobacteria (NTM)

1) target gene: 16S rRNA

2) primers

a. forward primer
NTM-1:
(SEQ ID NO: 38)
5′-tktggtggaaagcttttgc-3′
NTM-2:
(SEQ ID NO: 39)
5′-ggtgwgtggtgcaaagctt-3′
NTM-3:
(SEQ ID NO: 40)
5′-tggtggaaagcgtttggt-3′
b. reverse primer:
(SEQ ID NO: 21)
5′-cgtaggagtctgggccgta-3′

3) PCR product size: 152 bp

4) mean melting temperature of PCR product (Tm): 86.6±0.26° C.

2. Duplex PCR and Melting Profile Analysis

(1) Isolation of DNA DNA was isolated from 186 Mycobacterium species and 78 nontuberculous mycobacterium species, all recovered from clinical specimens, and from 68 standard ATCC mycobacteria species including M. abscessus ATCC 19977, M. acapulcensis KCTC 9501, M. africanum ATCC 25420, M. agri KCTC 9502, M. alvei KCTC 19709, M. asiaticum KCTC 9503, M. aurum KCTC 19457, M. austroafricanum KCTC 9504, M. avium ATCC 25291, M. bolletii KCTC 19281, M. botniense KCTC 19646, M. bovis ATCC 19210, M. brumae KCTC 19711, M. celatum ATCC 51131, M. chelonae subsp chelonae KCTC 9505, M. chlorophenolicum KCTC 19089, M. chubuense KCTC 19712, M. diernhoferi KCTC 9506, M. fallax KCTC 9508, M. flavescens ATCC 14474, M. fortuitum ATCC 6841, M. frederiksbergense KCTC 19100, M. gadium ATCC 27726, M. gastri ATCC 15754, M. gilvum KCTC 19423, M. goodii ATCC BAA-955, M. gordonae KCTC 9513, M. haemophilum ATCC 29548, M. hassiacum ATCC 700660, M. interjectum ATCC 51457, M. intermedium ATCC 51848, M. intracellulare ATCC 13950, M. intracellulare KCTC 9514, M. kansasii ATCC 12478, M. lentiflavum KMRC 70087, M. malmoense ATCC 29571, M. mantobense KCTC 9977, M. marinum ATCC 927, M. massiliense KCTC 19086, M. microti ATCC 19422, M. moriokaense KCTC 9516, M. mucogenicum KCTC 19088, M. neoaurum KCTC 19096, M. nonchromogenicum ATCC 19530, M. obuense KCTC 19097, M. parascrofulaceum KCTC 9979, M. peregrinum KCTC 9615, KMRC 75002, M. phlei KCTC 9689, M. porcinum KCTC 9517, M. pulveris KCTC 9518, M. scrofulaceum ATCC 19981, M. septicum ATCC 700731, M. simiae ATCC 25275, M. shimoidei ATCC 27962, M. smegmatis KCTC 9108, M. szulgai KCTC 9520, KMRC 31125, M. terrae KCTC 9614, M. triplex ATCC 700071, M. triviale KMRC 70093, M. tuberculosis ATCC 25177, ATCC 27294, M. ulcerans ATCC 19423, M. vaccae KCTC 19087, M. vanbaalenii KCTC 9966, M. wolinskyi ATCC 700010, and M. xenopi KMRC 42001.

The species identified in clinical subjects including 186 Mycobacterium species and 78 nontuberculous mycobacterium species were either detected in a liquid medium (MGIT mycobacterium medium) or a solid medium (Ogawa medium) or isolated directly from sputum specimens. The ATCC and KCTC standard species were cultured in broths while the KMRC species was grown on an agar plate.

From the mycobacteria cultured in broths, DNA was isolated as follows. Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

DNA was isolated from mycobacteria cultured on agar plates, as follows. One platinum loop taken from an agar plate was dissolved in 500 μL of sterile distilled water in a 1.5 mL tube, and heated for 10 min in a boiling water bath. Following centrifugation at 14,000 rpm for 5 min, the supernatant was used as a template in PCR.

Sputum specimens were treated as follows. One volume of 1 N NaOH was added to sputum in a 15 mL or 50 mL tube and left for min to liquify the sputum. Following centrifugation at 14,000 rpm for 2 min, the pellet was mixed well for 10 sec in 1 mL of sterile distilled water. Again, the mixture was centrifuged at 14,000 rpm for 2 min, and the pellet was mixed well for 10 sec in 1 mL of sterile distilled water. Centrifugation was performed at 14,000 rpm for 2 min, and the pellet was mixed well with 100 μL of 5% chelex resin (BioRad, USA) and 1 μL of 10 mg/mL proteinase K. After being left at 56° C. for 15 min, the mixture was heated for 10 min in a boiling water bath. Following centrifugation at 14,000 rpm for 5 min, the supernatant was taken for use as a template in PCR.

(2) Duplex PCR and Melting Profile Analysis

Duplex real-time PCR started with denaturation at 95° C. for 5 min and was run with 40 cycles of denaturation at 95° C. for 10 sec, annealing and extension at 60° C. for 30 sec, using a Type-it HRM PCR kit (QIAGEN Inc., Germantown, Md., USA) on Rotor-Gene Q(QIAGEN Inc., Germantown, Md., USA). This kit contained the fluorescent EvaGreen. After completion of the PCR, melting profile analysis was carried out by measuring absorbance at 510 nm while the PCR product was heated from 72° C. to 93° C. at an elevation rate of 0.2° C./sec. Because PCR depends on various factors including the concentrations of primers, the temperature and time of annealing and extension steps, the PCR conditions optimal for detecting both the target genes were determined in consideration of the factors. The composition of the duplex real-time PCR reagent is summarized in Table 3, below. In the primer mix, a forward primer and a reverse primer were contained in the same amounts (10 pmoles/μL). Accordingly, 1.875 μL of the primer mix for MTC contained the forward primer and the reverse primer in an amount of 18.75 pmoles, each. Since a total volume of PCR mixture was 25 μL, it contained the primers at a concentration of 0.75 μM (18.75 pmoles/25 μL). For NTM, each of the forward primer and the reverse primer was in an amount of 12.5 pmoles/1.25 μL, with a final concentration of 0.5 μM (12.5 pmoles/25 μL). In this context, the NTM-1 forward primer (SEQ ID NO: 38), the NTM-2 forward primer (SEQ ID NO: 39), and the NTM-3 forward primer (SEQ ID NO: 40) were used in substantially the same amounts. In the NTM-1 forward primer (SEQ ID NO: 38), 5′-tgtggtggaaagcttttgc-3′ (SEQ ID NO: 41) and 5′-tttggtggaaagcttttgc-3′ (SEQ ID NO: 42) were present at a ratio of about 1:1 while the NTM-2 forward primer (SEQ ID NO: 39) contained 5′-ggtgagtggtgcaaagctt-3′ (SEQ ID NO: 43) and 5′-ggtgtgtggtgcaaagctt-3′ (SEQ ID NO: 44) at a ratio of 1:1.

TABLE 3

| Ingredient | | Vol. (μL) | Conc. |
|---|---|---|---|
| 2X HRM PCR Master Mix | | 12.5 | 1X |
| Primer Mix (10 pmole/μL) | MTC | 1.875 | 0.75 uM |
| Primer Mix (10 pmole/μL) | NTM | 1.25 | 0.5 uM |

| | | |
|---|---|---|
| Nuclease free water | 4.375 | — |
| Sample DNA template | 5 | — |
| Total | 25 | — |

3. Result of Duplex Real-Time PCR and Melting Profile Analysis

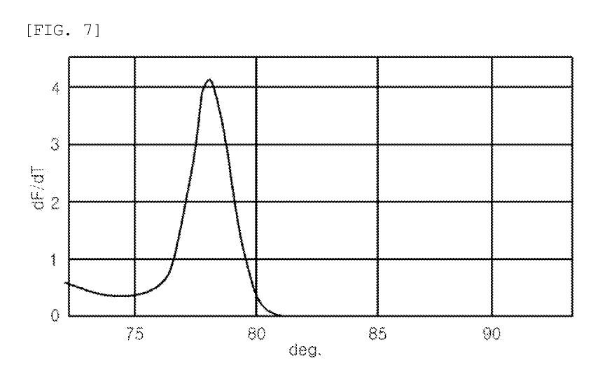

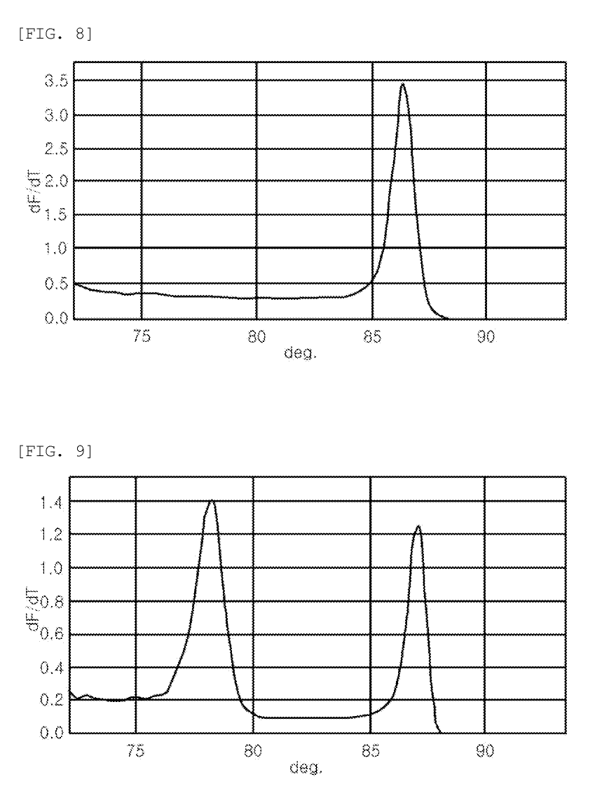

FIGS. 7 to 9 are melting profiles of the duplex real-time PCR products (amplicons) amplified from DNAs of the bacteria, that is, MTC, NTM, and MTC+NTM, respectively. In the profiles, temperatures (° C.) are set on the X-axis while a change in fluorescence intensity (F) per unit time (dF/dT) is on the Y-axis.

The MTC amplicon was measured to have a mean melting temperature (Tm) of about 78.0±0.20° C. while the Tm of the NTM amplicon was determined to be 86.6±0.26° C. For MTC+NTM, two respective Tm peaks were read.

Therefore, the duplex real-time PCR and melting profile analysis according to the present invention was found to guarantee the detection of MTC and NTM from clinical subjects, simultaneously, with high reliability.

In combination with the detection kits comprising forward and reverse primers designed on the basis of nucleotide sequences which are characteristic of MTC, or which are absent from MTC but intrinsic to NTM, as demonstrated in the Examples, duplex PCR and melting profile analysis can be used to detect MTC and NTM with high reliability. Therefore, the present invention provides a means for detecting MTC and NTM effectively.

Being capable of detecting nucleotide sequences characteristic of MTC and NTM, the primer sets of the present invention are highly sensitive to and selective for MTC and NTM when applied to detection, as described above. In addition, the duplex PCR using the primers of the present invention, and the melting profile analysis according to the present invention provide a clinical diagnostic means that promises the very effective, simultaneous detection of both MTC and NTM in a target subject.

### INDUSTRIAL APPLICABILITY

Useful in detecting genes characteristic of MTC and NTM, as described hitherto, the primer sets, detection kits, and detection methods according to the present invention can be applied to the clinical diagnosis of diseases caused by MTC and NTM, and therefore find applications in the medical fields including hospitals, research institutes, etc.

### Sequence List Text

The nucleotide sequence of SEQ ID NO: 1 is a forward primer specific for the IS6100 gene of Mycobacterium tuberculosis complex.

The nucleotide sequence of SEQ ID NO: 2 is a reverse primer specific for the IS6100 gene of Mycobacterium tuberculosis complex

The nucleotide sequence of SEQ ID NO: 3 is a forward primer specific for the 16S rRNA gene of nontuberculous mycobacteria.

The nucleotide sequence of SEQ ID NO: 4 is a reverse primer specific for the 16S rRNA gene of nontuberculous mycobacteria (NTM-1).

The nucleotide sequence of SEQ ID NO: 5 is a reverse primer specific for the 16S rRNA gene of nontuberculous mycobacteria (NTM-1).

The nucleotide sequence of SEQ ID NO: 6 is a reverse primer specific for the 16S rRNA gene of nontuberculous mycobacteria (NTM-1).

The nucleotide sequence of SEQ ID NO: 7 is a reverse primer specific for the 16S rRNA gene of nontuberculous mycobacteria (NTM-2).

The nucleotide sequence of SEQ ID NO: 8 is a forward primer specific for the 16S rRNA gene of nontuberculous mycobacteria.

The nucleotide sequence of SEQ ID NO: 9 is a forward primer specific for the 16S rRNA gene of nontuberculous mycobacteria.

The nucleotide sequence of SEQ ID NO: 10 is a forward primer specific for the 16S rRNA gene of nontuberculous mycobacteria.

The nucleotide sequence of SEQ ID NO: 11 is a forward primer specific for the 16S rRNA gene of nontuberculous mycobacteria.

The nucleotide sequence of SEQ ID NO: 12 is a forward primer specific for the 16S rRNA gene of nontuberculous mycobacteria.

The nucleotide sequence of SEQ ID NO: 13 is a forward primer specific for the 16S rRNA gene of nontuberculous mycobacteria.

The nucleotide sequence of SEQ ID NO: 14 is a forward primer specific for the 16S rRNA gene of nontuberculous mycobacteria.

The nucleotide sequence of SEQ ID NO: 15 is a forward primer specific for the 16S rRNA gene of nontuberculous mycobacteria.

The nucleotide sequence of SEQ ID NO: 16 is a reverse primer specific for the 16S rRNA gene of nontuberculous mycobacteria (NTM-2).

The nucleotide sequence of SEQ ID NO: 17 is a reverse primer specific for the 16S rRNA gene of nontuberculous mycobacteria (NTM-2).

The nucleotide sequence of SEQ ID NO: 18 is a forward primer specific for the IS6100 gene of Mycobacterium tuberculosis complex.

The nucleotide sequence of SEQ ID NO: 19 is a reverse primer specific for the IS6100 gene of Mycobacterium tuberculosis complex

The nucleotide sequence of SEQ ID NO: 20 is a forward primer specific for the 16S rRNA gene of nontuberculous mycobacteria.

The nucleotide sequence of SEQ ID NO: 21 is a reverse primer specific for the 16S rRNA gene of nontuberculous mycobacteria.

The nucleotide sequence of SEQ ID NO: 22 is a forward primer specific for the 16S rRNA gene of nontuberculous mycobacteria.

The nucleotide sequence of SEQ ID NO: 23 is a forward primer specific for the 16S rRNA gene of nontuberculous mycobacteria.

The nucleotide sequence of SEQ ID NO: 24 is a forward primer specific for the 16S rRNA gene of nontuberculous mycobacteria.

The nucleotide sequence of SEQ ID NO: 25 is a forward primer specific for the 16S rRNA gene of nontuberculous mycobacteria.

The nucleotide sequence of SEQ ID NO: 26 is a forward primer specific for the 16S rRNA gene of nontuberculous mycobacteria.

The nucleotide sequence of SEQ ID NO: 27 is a forward primer specific for the 16S rRNA gene of nontuberculous mycobacteria.

The nucleotide sequence of SEQ ID NO: 28 is a forward primer specific for the 16S rRNA gene of nontuberculous mycobacteria.

The nucleotide sequence of SEQ ID NO: 29 is a forward primer specific for the 16S rRNA gene of nontuberculous mycobacteria.

The nucleotide sequence of SEQ ID NO: 30 is a forward primer specific for the 16S rRNA gene of nontuberculous mycobacteria.

The nucleotide sequence of SEQ ID NO: 31 is a forward primer specific for the 16S rRNA gene of nontuberculous mycobacteria.

The nucleotide sequence of SEQ ID NO: 32 is a forward primer specific for the 16S rRNA gene of nontuberculous mycobacteria.

The nucleotide sequence of SEQ ID NO: 33 is a forward primer specific for the 16S rRNA gene of nontuberculous mycobacteria.

The nucleotide sequence of SEQ ID NO: 34 is a forward primer specific for the 16S rRNA gene of nontuberculous mycobacteria.

The nucleotide sequence of SEQ ID NO: 35 is a forward primer specific for the 16S rRNA gene of nontuberculous mycobacteria.

The nucleotide sequence of SEQ ID NO: 36 is a forward primer specific for the 16S rRNA gene of nontuberculous mycobacteria.

The nucleotide sequence of SEQ ID NO: 37 is a forward primer specific for the 16S rRNA gene of nontuberculous mycobacteria.

The nucleotide sequence of SEQ ID NO: 38 is a forward primer specific for the 16S rRNA gene of nontuberculous mycobacteria (NTM-1).

The nucleotide sequence of SEQ ID NO: 39 is a forward primer specific for the 16S rRNA gene of nontuberculous mycobacteria (NTM-2).

The nucleotide sequence of SEQ ID NO: 40 is a forward primer specific for the 16S rRNA gene of nontuberculous mycobacteria (NTM-3).

The nucleotide sequence of SEQ ID NO: 41 is a forward primer specific for the 16S rRNA gene of nontuberculous mycobacteria (NTM-1).

The nucleotide sequence of SEQ ID NO: 42 is a forward primer specific for the 16S rRNA gene of nontuberculous mycobacteria (NTM-1).

The nucleotide sequence of SEQ ID NO: 43 is a forward primer specific for the 16S rRNA gene of nontuberculous mycobacteria (NTM-2).

The nucleotide sequence of SEQ ID NO: 44 is a forward primer specific for the 16S rRNA gene of nontuberculous mycobacteria (NTM-2).

1. A primer set specific for the 16S rRNA gene of nontuberculous mycobacteria, comprising:
a forward primer having a nucleotide sequence of SEQ ID NO: 3;
at least one reverse primer selected from the group consisting of nucleotide sequences of SEQ ID NOS: 4 to 6; and
a reverse primer having a nucleotide sequence of SEQ ID NO: 7.

2. A kit for the detection of Mycobacterium tuberculosis and nontuberculous mycobacteria, comprising:
a primer set specific for the IS6110 gene of Mycobacterium tuberculosis, composed of a forward primer having a nucleotide sequence of SEQ ID NO: 1 and a reverse primer having a nucleotide sequence of SEQ ID NO: 2; and
a primer set specific for the 16S rRNA gene of nontuberculous mycobacteria, composed of a forward primer having a nucleotide sequence of SEQ ID NO: 3, at least one reverse primer selected from the group consisting of nucleotide sequences of SEQ ID NOS: 4 to 6, and a reverse primer having a nucleotide sequence of SEQ ID NO: 7.

3. A method for detecting Mycobacterium tuberculosis and nontuberculous mycobacteria, comprising:
isolating DNA from a test subject;
amplifying the DNA by duplex real-time PCR using a primer set specific for the IS6110 gene of Mycobacterium tuberculosis, composed of a forward primer having a nucleotide sequence of SEQ ID NO: 1 and a reverse primer having a nucleotide sequence of SEQ ID NO: 2; and a primer set specific for the 16S rRNA gene of nontuberculous mycobacteria, composed of a forward primer having a nucleotide sequence of SEQ ID NO: 3, at least one reverse primer selected from the group consisting of nucleotide sequences of SEQ ID NOS: 4 to 6, and a reverse primer having a nucleotide sequence of SEQ ID NO: 7;
heating the amplified DNA product over various temperature to produce a melting profile; and
analyzing the melting profile to determine a melting temperature of the target DNA.

4. A primer set specific for the 16S rRNA gene of nontuberculous mycobacteria, comprising a forward primer having a nucleotide sequence of SEQ ID NO: 20 and a reverse primer having a nucleotide sequence of SEQ ID NO: 21.

5. A kit for the detection of Mycobacterium tuberculosis and nontuberculous mycobacteria, comprising:
a primer set specific for the IS6110 gene of Mycobacterium tuberculosis, composed of a forward primer having a nucleotide sequence of SEQ ID NO: 18 and a reverse primer having a nucleotide sequence of SEQ ID NO: 19; and
a primer set specific for the 16S rRNA gene of nontuberculous mycobacteria, comprising a forward primer having a nucleotide sequence of SEQ ID NO: 20 and a reverse primer having a nucleotide sequence of SEQ ID NO: 21.

6. A method for detecting Mycobacterium tuberculosis and nontuberculous mycobacteria, comprising:
isolating DNA from a test subject;
amplifying the DNA by duplex real-time PCR using a primer set specific for the IS6110 gene of Mycobacterium tuberculosis, composed of a forward primer having a nucleotide sequence of SEQ ID NO: 18 and a reverse primer having a nucleotide sequence of SEQ ID NO: 19; and a primer set specific for the 16S rRNA gene of nontuberculous mycobacteria, comprising a forward primer having a nucleotide sequence of SEQ ID NO: 20 and a reverse primer having a nucleotide sequence of SEQ ID NO: 21;
heating the amplified DNA product over various temperature to produce a melting profile; and
analyzing the melting profile to determine a melting temperature of the target DNA.

7. A primer set specific for the 16S rRNA gene of nontuberculous mycobacteria, comprising:
a forward primer composed of respective primers having the nucleotide sequences of SEQ ID NOS: 38 to 40; and
a reverse primer having a nucleotide sequence of SEQ ID NO: 21.

8. A kit for the detection of Mycobacterium tuberculosis and nontuberculous mycobacteria, comprising:
a primer set specific for the IS6110 gene of Mycobacterium tuberculosis, composed of a forward primer having a nucleotide sequence of SEQ ID NO: 18 and a reverse primer having a nucleotide sequence of SEQ ID NO: 19; and
a primer set specific for the 16S rRNA gene of nontuberculous mycobacteria, comprising a forward primer composed of respective primers having the nucleotide sequences of SEQ ID NOS: 38 to 40, and a reverse primer having a nucleotide sequence of SEQ ID NO: 21.

9. A method for detecting Mycobacterium tuberculosis and nontuberculous mycobacteria, comprising:
isolating DNA from a test subject;
amplifying the DNA by duplex real-time PCR using a primer set specific for the IS6110 gene of Mycobacterium tuberculosis, composed of a forward primer having a nucleotide sequence of SEQ ID NO: 18 and a reverse primer having a nucleotide sequence of SEQ ID NO: 19; and a primer set specific for the 16S rRNA gene of nontuberculous mycobacteria, comprising a forward primer composed of respective primers having the nucleotide sequences of SEQ ID NOS: 38 to 40, and a reverse primer having a nucleotide sequence of SEQ ID NO: 21;
heating the amplified DNA product over various temperature to produce a melting profile; and
analyzing the melting profile to determine a melting temperature of the amplified product.
